(19) United States
(12) Patent Application Publication
(10) Pub. No.: US 2004/0091555 A1
(43) Pub. Date: May 13, 2004

(54) Methods for producing agrimonia extracts with improved activity against hepatitis b virus and pharmaceutical and food compositions containing said extracts

(76) Inventors: Young-Sung Lee, Seoul (KR); Dong-Ho Ahn, Gyeonggi-do (KR); Sa-Min Hong, Seoul (KR); Seung-Kew Yoon, Seoul (KR); Eun-Joo Chang, Daejeon (KR); Min-Kyung Lee, Gyeonggi-do (KR); Hyok-Yun Kwon, Daejeon (KR)

Correspondence Address: HUNTON & WILLIAMS LLP, 1601 BRYAN STREET, ENERGY PLAZA - 30TH FLOOR, DALLAS, TX, 75201, US

(21) Appl. No.: 10/296,753

(22) PCT Filed: Oct. 9, 2002
(86) PCT No.: PCT/KR02/01888

(51) Int. Cl. A61K 35/78

(52) U.S. Cl. 424/725

(57) ABSTRACT

Disclosed are pharmaceutical and food compositions for preventing or treating hepatic disease, containing a pharmaceutically effective amount of an extract from a plant Agrimonia eupatoria L. Also, the present invention discloses a method of preparing a water-soluble extract from a plant of the genus Agrimonia, which which has improved efficacy in preventing or treating hepatic diseases, characterized by heating a mixture of a pulverized product of a plant of the genus Agrimonia and water at about 20° C. to 30° C. for about 7 days or more, and pharmaceutical and food compositions for preventing or treating a hepatic disease, containing a water-soluble extract prepared according to the method. Further, the present invention discloses a method of preparing an organic solvent-soluble extract from a plant of the genus Agrimonia, which has improved efficacy in preventing or treating hepatic diseases, comprising the steps of extracting a plant of the genus Agrimonia with an organic solvent, and removing an aqueous fraction and then obtaining an organic solvent-soluble fraction, and pharmaceutical and food compositions for preventing or treating hepatic disease, containing an extract prepared according to the method.

### TECHNICAL FIELD

The present invention relates to methods of preparing extracts from plants belonging to the genus Agrimonia, which have improved activity against Hepatitis B virus. Also, the present invention is concerned with pharmaceutical and food compositions containing such extracts, which are useful for preventing and treating hepatic diseases.

### BACKGROUND ART

It is known that hepatitis B virus (HBV), which is a DNA virus, causes chronic hepatitis as well as acute hepatitis in humans, and is thus a major target of clinical research. When chronic hepatitis persists for a long period of time, liver cirrhosis and hepatocellular carcinoma can be induced (Peter J. Grob. Vaccine, 16:S11-S16 (1998)). Most hepatitis B viruses infect hosts via parenteral routes. In particular, infection of infants by HBV is a major global concern. If the mother has a chronic HBV infection, a fetus or a newborn infant is exposed to infection with HBV transmitted from the mother. Over two hundred million-people in the world suffer from HBV infection, and 3,500,000 people among those are in states of chronic infection which progress to liver cirrhosis and hepatocellular carcinoma, resulting in a high mortality rate (bavey S. State of the World's Vaccines and Immunization. Geneva: WHO, 1996:76-82). However, there is now no effective therapeutic agent for treatment of HBV infection.

In this regard, a large number of research efforts for preventing or treating HBV infection are underway. As a result, the developed therapeutic agents are classified into immunomodulators, antiviral agents and nucleoside analogues, depending on their properties. The representative example of the immunomodulators is interferon, and examples of the antiviral agents include ribavirin, vidarabine, ara-AMP, acyclovir, suramin and zidovudine. Also, the nucleoside analogues include lamivudine, and adefovir having recently received approval from the Food and Drug Administration (FDA) in the USA for treatment of hepatitis B.

Of these, interferon (INF) has been actively studied for its therapeutic effects in chronic hepatitis patients since Greenberg H. B. reported that leukocytic IFN has inhibitory activity against proliferation of HBV (Greenberg H. B. et al., N Engl J Med, 295:517-522 (1976)). In its early stages, clinical research on INF was limited owing to insufficient supply and undefined dosage and duration of treatment. With development of recombination techniques, which facilitated use of IFN, IFN alpha-2b (hereinafter, referred to as “α-IFH”) is now commercially available, and has been approved as a drug for therapy of chronic hepatitis B. However, patients with chronic hepatitis B display a low reactivity of below 25% for α-IFN. β-IFN and γ-IFN have therapeutic efficacy only in patients with HBV infection of less than 4 months.

Corticosteroids were proved to have effectiveness versus autoimmune hepatitis, and thus were studied for use in treating chronic hepatitis B (Marsha A. Viral Hepatitis. In: Dipiro J T et al., eds. Pharmacotherapy: a pathophysiologic approach. 3^{rd} ed. New York: Elservier, 829-852 (1996)). In spite of having an effect of reducing liver inflammation in most hepatitis patients, corticosteroids allowed rapidly increased replication of HBV Thus, when stopping corticosteroid therapy, the inflammatory activity is elevated. In addition, corticosteroids are known to worsen the pathogenic state of hepatitis patients, thus limiting their application range.

The antiviral drug ribavirin is efficacious for treatment of chronic hepatitis B, and advantageous in terms of being orally administrable. However, when administration of ribavirin was stopped, all experiments showed a high recurrence in HBV infection. Vidarabine and its derivative ara-AMP do not display continuous pharmaceutical effects and cause severe neuromuscular toxicity. For these reasons, vidarabine and ara-AMP are not applied for treatment of hepatitis. Several other antiviral agents have latent toxicity because of negatively affecting cellular nucleic DNA and mitochondrial DNA, as well as viral DNA, and having limited amount of clinical data.

Recently, drugs derived from nucleic acids have been developed as new therapeutic agents, for treating hepatitis B patients, which are exemplified as famcyclovir, lamivudine, lobucavir and adefovir dipivoxil (Erik De Clercq, Int J Antimicrob Agent, 12:81-95 (1999)). At the early stages, such dideoxy nucleoside analogues (ddNs) derived from HIV therapeutic agents, which have an effect of suppressing reverse transcriptase, or therapeutic agents for some herpes virus infections were developed for treatment of other viral infections, and were demonstrated to be also effective in treating HBV infection. From very extensive studies on famcyclovir and lamivudine, it is reported that the two drugs can be administered orally. In famcyclovir or lamivudine therapy, it is reported that in some patients, serum HBV-DNA levels decreased rapidly, HbeAg (an envelope antigen of HBV) disappeared, and liver-specific enzyme levels were normalized. In the preliminary clinical application of lamivudine to hepatitis B patients for one year, it was observed that an HBeAg seroconversion rate to HBeAb was as low as 22%, while alanine transaminase (ALT) and aspartate transaminase (AST) levels are normalized. However, when stopping the lamivudine therapy, in most cases, inflammation recurred and liver enzymes levels were increased, and HBV mutant strains resistant to lamivudine emerged within several months. For the past year, clinical data has shown that long-term lamivudine therapy causes emergence of drug resistant HBV mutants in about 14% of patients after several months of lamivudine therapy, and in about 30% of patients after two years (Alen M I, Hepatology, 27:1670-1677 (1998)). In particular, emergence of drug resistant HBV mutant strains has been reported in liver transplant patients with HBV infection (Yao F Y, Liver Transplant Surgery, 5:491-496 (1999)). Resistant HBV mutant strains harbor point mutations in the HBV polymerase gene, predominantly in the well-conserved YMDD motif, in which YMDD mutation gives HBV resistance to lamivudine, while sustaining viral replication. Also, liver transplantation, which is conducted as a therapy for HBV infection, is disadvantageous in terms of having high recurrence of HBV infection.

On the other hand, components isolated from plants, including glycyrrhizine, catechin and silymarin, are known to have an inhibitory effect against hepatitis viruses. Despite having a suppressive effect on replication of hepatitis viruses and good liver protection function (Altern Med Rev 1999 August; 4(4):220-38, Zhongguo Zhong Xi Yi Jie He Za Zhi 1992 August; 12(8):480-2), such components are still not developed as therapeutic agents for hepatitis.

Also, many chemotherapeutic agents known to be clinically useful for treatment of various cancers have been demonstrated to be ineffective against hepatocellular carcinoma. As a representative example, paclitaxel having a suppressive activity against some malignant tumor cells, which is isolated from Taxus cuspidate, was demonstrated to have no significant efficacy on hepatocellular carcinoma in phase II clinical trials using patients with hepatocellular carcinoma (British Journal of Cancer, 78(1), 34-39, 1998)). Materials with activity versus hepatocellular carcinoma have been reported in various journals. For example, an octapeptide derivative of somatostatin has a therapeutic effect versus hepatoma, as disclosed in U.S. Pat. No. 5,411,943. U.S. Pat. No. 5,981,774 discloses some gamma-pyrone compounds, cyclomorusin, cycloartomunin, dihydrocyclo-artomunin, artomunoxanth-otrione epoxide, dihydroisocycloartomunin, artomunoxanthone, cyclocommunol, cyclo-mulberrin, and cyclocommunin, which are isolated from the root bark of Formosan tripterospermum plants and have exhibited cytotoxic effects against human hepatoma. Cytarabine ocfosfate and docetaxel, which are effective in treating hepatoma, are disclosed in European Pat. No. 0652469A1 and International Pat. Pub. No. WO 01/15675, respectively. Also, Korean Pat. No. 10-0187881 discloses a therapeutic agent for hepatocellular carcinoma containing decursinol angelate as an effective ingredient, and Korean Pat. Laid-open Publication No. 2000-0046779 discloses a composition for treating hepatitis or hepatoma containing an extract from a plant Urtica dioca L. as an effective ingredient.

There is still a need for further development of effective and improved therapeutic agents for treating hepatitis and hepatoma, which is also an object of the present invention. The present inventors previously found therapeutic agents for liver-related diseases containing an extract from a plant Lepidium apetalum W or Agrimonia pilosa L. as an effective component, which has a suppressive activity against production of surface antigens of HBV, as described in Korean Pat. Nos. 10-0351755 and 10-0327762.

### DISCLOSURE OF INVENTION

Conducted by the present inventors, the intensive and thorough research into therapeutic effects of extracts isolated from various species of the genus Agrimonia versus hepatic diseases resulted in the finding that, when added to cell cultures typically used for estimating efficacy of agents for treating hepatitis B virus (HBV), an extract from Agrimonia eupatoria L. of the genus Agrimonia has a very high effectiveness as a therapeutic agent for treatment of hepatic diseases by significantly suppressing production of surface antigens and envelope antigens of HBV, replication of HBV itself, and the activity of viral DNA polymerase participating in the replication of HBV. In addition, the present inventors discovered that a water-soluble extract from a plant of the genus Agrimonia, which is prepared by mixing the whole of a plant belonging to the genus Agrimonia with water and then heating the mixture at about 55° C. or more, has significantly increased efficacy in treating hepatic diseases, and that a water-soluble extract from a plant of the genus Agrimonia, prepared by mixing the whole of a plant of the genus Agrimonia with water and then heating the mixture at about 20° C. to 30° C. for about 7 days or more, has significantly increased efficacy in treating hepatic diseases. Further, the present inventors discovered that an organic solvent-soluble extract, prepared by extracting the whole of a plant of the genus Agrimonia in an organic solvent, is more effective in treating hepatic diseases than the water-soluble extracts.

Therefore, in an aspect of the present invention, there is provided a pharmaceutical composition for preventing or treating hepatic diseases, comprising a pharmaceutically effective amount of an extract from the plant Agrimonia eupatoria L. as an effective ingredient.

In another aspect of the present invention, there is provided a food composition for patients suffering from hepatic diseases, comprising an extract from the plant Agrimonia eupatoria L. as an effective ingredient.

In still another aspect of the present invention, there is provided a method of preparing a water-soluble extract from the plant Agrimonia eupatoria L., which is effective in preventing or treating hepatic diseases, comprising the steps of pulverizing a plant Agrimonia eupatoria L., mixing the pulverized product with water, heating the mixture to give an extract solution, and removing water-insoluble components from the extract solution.

In a further aspect of the present invention, there is provided a method of preparing a water-soluble extract from a plant of the genus Agrimonia, which is effective in preventing or treating hepatic diseases, comprising the steps of pulverizing a plant of the genus Agrimonia, mixing the pulverized product with water, heating the mixture at about 55° C. or more to give an extract solution, and removing water-insoluble components from the extract solution to produce a water-soluble extract.

In a still further aspect of the present invention, there is provided a pharmaceutical composition for preventing or treating hepatic diseases, containing a water-soluble extract from a plant of the genus Agrimonia, prepared by pulverizing a plant of the genus Agrimonia, mixing the pulverized plant with water, heating the mixture at about 55° C. or more to give an extract solution, and removing water-insoluble components from the extract solution to produce a water-soluble extract.

In a still further aspect of the present invention, there is provided a food composition for patients suffering from hepatic diseases, containing a water-soluble extract from a plant of the genus Agrimonia, prepared by pulverizing a plant of the genus Agrimonia, mixing the pulverized product with water, heating the mixture at about 55° C. or more to give an extract solution, and removing water-insoluble components from the extract solution to produce a water-soluble extract.

In a still further aspect of the present invention, there is provided a method of preparing a water-soluble extract from a plant of the genus Agrimonia, which has improved efficacy in preventing or treating hepatic diseases, comprising the steps of pulverizing a plant of the genus Agrimonia, mixing the pulverized product with water, heating the mixture at about 20 to 30° C. for about 7 days or more to give an extract solution, and removing water-insoluble components from the extract solution to produce a water-soluble extract.

In a still further aspect of the present invention, there is provided a pharmaceutical composition for preventing or treating hepatic diseases, containing a water-soluble extract from a plant of the genus Agrimonia, prepared by pulverizing a plant of the genus Agrimonia, mixing the pulverized product with water, heating the mixture at about 20 to 30° C. for about 7 days or more to give an extract solution, and removing water-insoluble components from the extract solution to produce an water-soluble extract.

In a still further aspect of the present invention, there is provided a food composition for patients suffering from hepatic diseases, containing a water-soluble extract from a plant of the genus Agrimonia, prepared by pulverizing a plant of the genus Agrimonia, mixing the pulverized product with water, heating the mixture at about 20 to 30° C. for about 7 days or more to give an extract solution, and removing water-insoluble components from the extract solution to produce a water-soluble extract.

In a still further aspect of the present invention, there is provided a method of preparing an organic solvent-soluble extract from a plant of the genus Agrimonia, comprising the steps of extracting a plant of the genus Agrimonia with an organic solvent, and removing an aqueous fraction and then obtaining an organic solvent-soluble fraction, which has improved efficacy in preventing or treating hepatic diseases.

In a still further aspect of the present invention, there is provided a pharmaceutical composition for preventing or treating hepatic diseases, containing an organic solvent-soluble extract from a plant of the genus Agrimonia, prepared by extracting a plant of the genus Agrimonia with an organic solvent, and removing an aqueous fraction and then obtaining an organic solvent-soluble fraction.

In a still further aspect of the present invention, there is provided a food composition for patients suffering from hepatic diseases, containing an organic solvent-soluble extract from a plant of the genus Agrimonia, prepared by extracting a plant of the genus Agrimonia with an organic solvent, and removing an aqueous fraction and then obtaining an organic solvent-soluble fraction.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a flowchart showing a method of obtaining an organic solvent-soluble extract with anti-HBV activity by treating a plant Agrimonia eupatoria L. with an organic solvent;

FIG. 2 is a graph showing an inhibitory activity of a water-soluble extract from a plant Agrimonia eupatoria L. against surface antigens of HBV (HBsAg) in PLC/PRF/5 cell lines;

FIG. 3 is a graph showing an inhibitory activity of a water-soluble extract from a plant Agrimonia eupatoria L. against envelope antigens of HBV (HBeAg) in HepG2 2.2.15 cell lines;

FIG. 4 is a result of an agarose gel electrophoresis showing an inhibitory activity of a water-soluble extract from Agrimonia eupatoria L. against HBV replication in HepG2 2.2.15 cell lines;

FIG. 5 is a result of an agarose gel electrophoresis showing an inhibitory activity of a water-soluble extract from Agrimonia eupatoria L. against HBV DNA polymerase in hepatitis patients;

FIG. 6 is a graph showing inhibitory activities of water-soluble extracts from Agrimonia eupatoria L., Agrimonia pilosa L., Agrimonia coreana N., and Agrimonia coreana N. for. pilosella Satake against surface antigens of HBV (HBsAg) in PLC/PRF/5 cell line;

FIG. 7 is a graph showing inhibitory activities of water-soluble extracts from Agrimonia eupatoria L., extracted at various temperatures for the same period of time (1 hr), against production of surface antigens of HBV (HBsAg) in PLC/PRF/5 cell line;

FIG. 8 is a graph showing inhibitory activities of water-soluble extracts from a plant Agrimonia eupatoria L., extracted at the same temperature (25° C.) for various periods of time, against production of surface antigens of HBV (HBsAg) in PLC/PRF/5 cell line;

FIG. 9 is a graph showing an inhibitory effect of fractions, obtained by drying and pulverizing the whole of a plant Agrimonia eupatoria L., and extracting a mixture of the pulverized product and distilled water with an equivalent volume of a water-insoluble organic solvent, hexane, chloroform or butylalcohol, against production of surface antigens of HBV (HBsAg) in HepG2 2.2.15 cell line;

FIG. 10 is a graph showing an inhibitory effect of fractions, obtained by drying and pulverizing the whole of Agrimonia eupatoria L., and extracting a mixture of the pulverized product and distilled water with an equivalent volume of a water-insoluble organic solvent, hexane, chloroform or butylalcohol, against production of envelope antigens of HBV (HBeAg) in HepG2 2.2.15 cell line; and

FIG. 11 is a flowchart showing a process of preparing a water-soluble extract with anti-HBV activity from the whole of Agrimonia eupatoria L.

### BEST MODE FOR CARRYING OUT THE INVENTION

Some plants belonging to the genus Agrimonia are known to contain components having an antitumor effect and an immunomodulating effect. Of the components, agrimoniin is a tannin contained in Agrimonia pilosa L., and acts as an immunomodulator by enhancing immune response, as well as an antitumor agent by inducing interleukin-1 production. Also, extracts from Agrimonia eupatoria L. are effective in treating diabetes. However, there are no reports describing extracts from plants of the genus Agrimonia as having an inhibitory effect against HBV, except a patent describing a water-soluble extract from Agrimonia pilosa L. having an inhibitory effect against production of surface antigens of HBV, as disclosed in Korea Pat. No 10-0327762.

Upon developing a therapeutic agent, evaluation of its in vitro and/or in vivo efficacy is of importance. Chimpanzees or woodchucks are typically used as in vivo models (Raymond F Schinazi, Antiviral Chem & Chemother, 10:99-114 (1999)), and cell lines derived from human hepatic tumor cells are used for in vitro studies, including HepG2 2.2.15 cell line from hepatic tumor cells HepG2, and Hep3B and PLC/PRF/5 cell lines from hepatic tumor tissue. HepG2 cells do not carry the HBV genome. In contrast, PLC/PRF/5 and HepG2 2.2.15 cells harbor the HBV genome in their genomic DNA (Mary A. Sells, P.N.A.S. 84:1005-1009 (1987)). Such cell lines, PLC/PRF/5 and HepG2 2.2.15 cells, like hepatic cells infected with HBV, have the ability to produce surface antigens and envelope antigens of HBV and HBV virions, and thus are useful for evaluating efficacy of therapeutic agents against HBV infection.

Plants belonging to the genus Agrimonia, useful in the present invention, include the following species: Agrimonia eupatoria L.; Agrimonia pilosa L.; Agrimonia coreana N.; Agrimonia coreana N. for. pilosella Satake; Agrimonia gryposepala; Agrimonia rostellata; Agrimonia pubescens; Agrimonia parviflora; Agrimonia striata; Agrimonia ordorata; Agrimonia incisa; Agrimonia polyphylla; Agrimonia microcarpa; Agrimonia bracteata; Agrimonia repens; Agrimonia platycarpa; Agrimonia pumila; and Agrimonia asiatica.

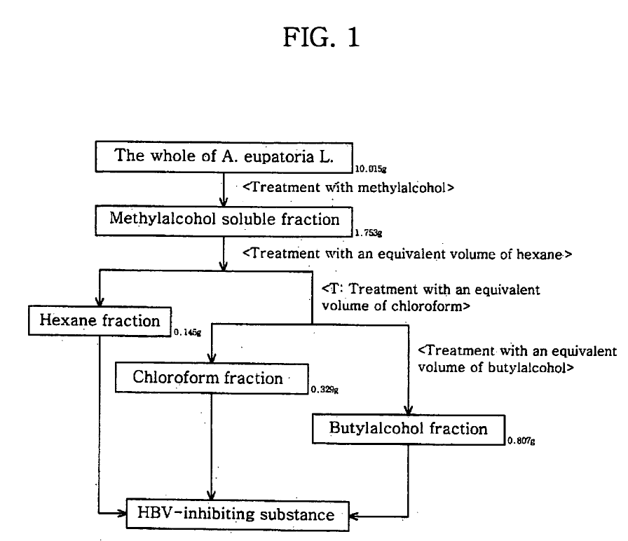

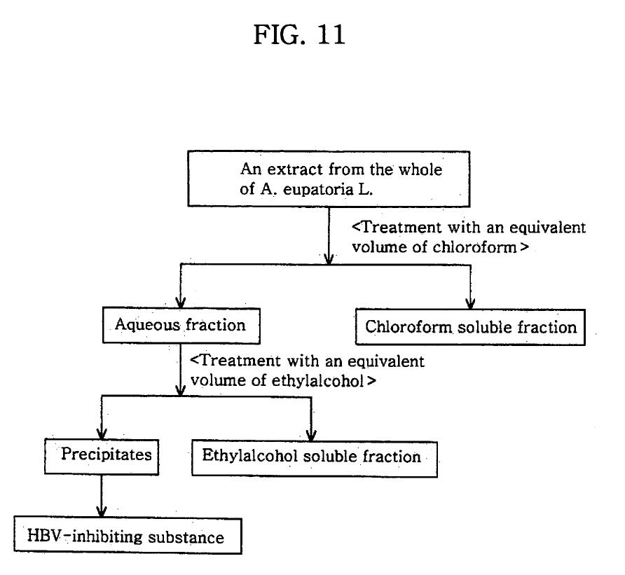

An extract from a plant of the genus Agrimonia according to the present invention is prepared according to any one of the following three methods. According to a first method, a water-soluble extract is prepared by mixing the plant with water and then heating the mixture. According to a second method, as showed in FIG. 11, a water-soluble extract is prepared by extracting the plant with an organic solvent and then removing an organic solvent-soluble fraction from the extract. Through a third process, as shown in FIG. 1, an organic solvent-soluble fraction is prepared by extracting the plant with an organic solvent and then removing an aqueous fraction from the extract. In accordance with the present invention, when being added to cells capable of evaluating efficacy of therapeutic agents versus HBV, all of the extracts have inhibitory effects against production of surface antigens and envelope antigens of HBV, HBV replication, and the activity of HBV DNA polymerase. In particular, extracts from Agrimonia eupatoria L. have about 1.6 to 2 times higher efficacy than extracts from other species of the genus Agrimonia, in inhibiting the production of surface antigens and envelope antigens of HBV, HBV replication, and the activity of HBV DNA polymerase. Therefore, the present invention provides a pharmaceutical composition for preventing or treating hepatic diseases, containing a pharmaceutically effective amount of an extract from Agrimonia eupatoria L. In another aspect of the present invention, there is provided a food composition for patients suffering from hepatic diseases, containing an extract from Agrimonia eupatoria L. as an effective ingredient. In still another aspect of the present invention, there is provided a method of preparing a water-soluble extract from a plant Agrimonia eupatoria L., comprising the steps of pulverizing a plant Agrimonia eupatoria L., mixing the pulverized product with water, heating the mixture to obtain an extract solution, and removing water-insoluble components from the extract solution, the water-soluble extract having improved efficacy in preventing or treating hepatic diseases.

In accordance with the present invention, a water-soluble extract from a plant of the genus Agrimonia, in detail, is prepared according to a method comprising the steps of drying and pulverizing the whole of a plant of the genus Agrimonia, suspending the pulverized resultant in distilled water, heating the suspension, and removing water-insoluble components from the suspension by centrifugation, and may be used in a liquid or dried form. In an embodiment of the present invention, temperature and duration of the heating significantly affects efficacy of the final water-soluble extract, in which a water-soluble extract prepared by heating at about 55° C. or more has remarkably higher efficacy. Therefore, the present invention provides a method of preparing a water-soluble extract from a plant of the genus Agrimonia, which has improved efficacy in preventing or treating hepatic diseases, comprising the steps of pulverizing a plant of the genus Agrimonia, mixing the pulverized product with water, heating the mixture at about 55° C. or more to obtain an extract solution, and removing water-insoluble components from the extract solution. Also, the present invention provides a pharmaceutical composition for preventing or treating hepatic diseases, containing a water-soluble extract from a plant of the genus Agrimonia, prepared by pulverizing a plant of the genus Agrimonia, mixing the pulverized product with water, heating the mixture at about 55° C. or more to obtain an extract solution, and removing water-insoluble components from the extract solution to produce a water-soluble extract. Further, the present invention provides a food composition for patients suffering from hepatic diseases, containing a water-soluble extract from a plant of the genus,Agrimonia, prepared by pulverizing a plant of the genus Agrimonia, mixing the pulverized product with water, heating the mixture at about 55° C. or more to obtain an extract solution, and removing water-insoluble components from the extract solution to produce a water-soluble extract.

In an embodiment of the present invention, when being prepared by heating at about 20 to 30° C. for about 7 days or more, efficacy of the water-soluble extract has a remarkably improved efficacy versus hepatic diseases. Therefore, the present invention provides a method of preparing a water-soluble extract from a plant of the genus Agrimonia, which has improved efficacy in preventing or treating hepatic diseases, comprising the steps of pulverizing a plant of the genus Agrimonia, mixing the pulverized product with water, heating the mixture at about 20 to 30° C. for about 7 days or more to obtain an extract solution, and removing water-insoluble components from the extract solution to produce a water-soluble extract: Also, the present invention provides a pharmaceutical composition for preventing or treating hepatic diseases, containing a water-soluble extract from a plant of the genus Agrimonia, prepared by pulverizing a plant of the genus Agrimonia, mixing the pulverized product with water, heating the mixture at about 20 to 30° C. for about 7 days or more to obtain an extract solution, and removing water-insoluble components from the extract solution to produce a water-soluble extract. Further, the present invention provides a food composition for patients suffering from hepatic diseases, containing a water-soluble extract from a plant of the genus Agrimonia, prepared by pulverizing a plant of the genus Agrimonia, mixing the pulverized product with water, heating the mixture at about 20 to 30° C. for about 7 days or more to obtain an extract solution, and removing water-insoluble components from the extract solution to produce a water-soluble extract.

In accordance with the present invention, an extract from a plant of the genus Agrimonia is, in detail, prepared according to a method comprising the steps of extracting the whole of a plant of the genus Agrimonia with an organic solvent such as methylalcohol, treating with a water-insoluble organic solvent to separate the extract into an organic solvent fraction and an aqueous fraction, and treating the aqueous fraction with another water-insoluble organic solvent, resulting in formation of separate organic solvent fractions and a final aqueous fraction. In an embodiment of the present invention, organic solvent fractions from plants of the genus Agrimonia are about 1.5 to 3 times more effective than the final aqueous fraction in inhibiting the production of surface antigens and envelope antigens of HBV, HBV replication and the activity of HBV DNA polymerase. Therefore, the present invention provides a method of preparing an organic solvent-soluble extract from a plant of the genus Agrimonia, comprising the steps of extracting a plant of the genus Agrimonia with an organic solvent, and removing an aqueous fraction and then obtaining an organic solvent-soluble fraction, which has improved efficacy in preventing or treating hepatic diseases. Also, the present invention provides a pharmaceutical composition for preventing or treating hepatic diseases, containing an organic solvent-soluble extract from a plant of the genus Agrimonia, prepared by extracting a plant of the genus Agrimonia with an organic solvent, and removing an aqueous fraction and then obtaining an organic solvent-soluble fraction. Further, the present invention provides a food composition for patients suffering from hepatic diseases, containing an organic solvent-soluble extract from a plant of the genus Agrimonia, prepared by extracting a plant of the genus Agrimonia with an organic solvent, and removing an aqueous fraction and then obtaining an organic solvent-soluble fraction.

Organic solvents useful in the present invention, but are not limited to, include alcohols, which are exemplified as ethanol, methanol, butylalcohol, isopropanol and ethylene glycol, halogen-substituted hydrocarbons, which are exemplified by methylene chloride, chloroform and carbon tetrachloride, tetrahydrofuran, hexane, DMF (N,N-dimethylformamide), DMSO (dimethylsulfoxide), and ethylacetate.

Hepatitis B virus (HBV), which is a DNA virus, is of clinical importance as a cause of chronic hepatitis as well as acute hepatitis in humans. When chronic hepatitis persists for a long period of time, liver cirrhosis and hepatocellular carcinoma can be induced (Peter J. Grob. Vaccine, 16:S11-S16 (1998)). Because of having excellent effects of inhibiting production of surface antigens and envelope antigens of HBV, HBV replication and the activity of HBV DNA polymerase, the extract from a plant of the genus Agrimonia according to the present invention is very useful for preventing and treating liver cirrhosis and hepatocellular carcinoma, as well as hepatitis.

In addition, the extract from a plant of the genus Agrimonia according to the present invention may be administered alone or in combination with a pharmaceutically acceptable carrier, diluent or exipient, as a pharmaceutical composition, in a pharmaceutically effective amount. The term “a pharmaceutically effective amount”, as used herein, refers to an amount sufficient for treatment or prevention of diseases, which is commensurate with a reasonable benefit/risk ratio applicable for medical treatment or prevention. However, it will be appreciated that a medical doctor can determine a dosage per day of the Agrimonia extract and its pharmaceutical compositions using his/her reasonable medical judgment. An effective dosage amount of the Agrimonia extract and its pharmaceutical compositions for a patient may be determined depending on the patient's diseases and pathogenic states; the activity of an extract to be used; a composition to be used; the patient's age, body weight, health state, sex and diet; administration time, administration route and an excretion rate of a used extract; duration of treatment; drugs used in combination with or simultaneously used with a used extract; and other factors known in medical fields. For example, it is well known to those of ordinary skill in the art that the extract according to the present invention should be administered starting at a level lower than a required level and then gradually increased in dosage until achieving a desired therapeutic result.

For an adult of 60 kg body weight, an effective dosage of a pharmaceutically effective amount of the Agrimonia extract is preferably 10 ml in a liquid form given 3 times daily for at least 12 weeks, or 20 mg dry weight in a tablet or capsule form given 3 times daily for at least 12 weeks, and such an administration may be conducted repeatedly according to intended use. A unit dosage of the extract may be changed properly according to a patient's age, body weight, health state, sex, pathogenic state and diet, the extract's administration time, administration route and an excretion rate, and the like.

The Agrimonia extract according to the present invention can be administered via a typical route, for example, in an oral dosage form, such as tablets, capsules, sugar-coated tablets or film-coated tablets, liquid solutions, or suspensions, or in a non-oral dosage form, such as rectal suppositories, or intramuscular, intravenous and/or intrathecal and/or intraspinal injections or infusions.

The Agrimonia extract according to the present invention is provided as a pharmaceutical composition in combination with a pharmaceutically acceptable carrier, diluent or exipient, which contains a specific amount of the Agrimonia extract. The pharmaceutical composition may be formulated into various pharmaceutically administrable forms by conventional methods, and administered in a pharmaceutically suitable form. For example, solid formulations for oral administration can contain a diluent (e.g., lactose, dextrose, sucrose, cellulose, corn starch or potato starch), a lubricant (e.g., silica, talc, stearic acid, magnesium or calcium stearate, and/or polyethylene glycol), a binder (e.g., starch, arabic gum, methycellulose gelatin, carboxymethylcellulose or polyvinylpyrrolidone), a disintegrator (e.g., starch, arginic acid, arginate or sodium starch glycolate), a dye, a sweetener, a humectant (e.g., lecitin, polysorbate, laurylsulfate) and other pharmacokinetically inactive materials which are commonly used in the art. Pharmaceutical formulations of the Agrimonia extract can be prepared by conventional methods well known to those of ordinary skill in the art, for example, by performing a process comprising the steps of mixing, granulating, forming into tablets, and coating with sugar or film.

As another way for oral administration of the Agrimonia extract according to the present invention, the Agrimonia extract may be formulated into liquid dispersions, which are typically syrups, emulsions and suspensions. Emulsions and suspensions can contain a carrier, which is exemplified as natural gum, agarose, sodium arginate, pectin, methylcellulose, carboxymethylcellulose or polyvinyl alcohol.

Suspensions or solutions for intramuscular injection of the Agrimonia extract according to the present invention can contain a pharmaceutically acceptable carrier, which is exemplified as sterilized water, olive oil, ethyl oleate, glycol (e.g., propylene glycol), along with an active ingredient, and a,suitable amount of lidocaine hydrochloride according to intended use. Solutions for intravenous injections or infusions of the Agrimonia extract can contain sterile water as a carrier, preferably sterile isotonic saline, or a carrier propylene glycol.

Suppositories for administration of the Agrimonia extract can, along with an active ingredient, contain a pharmaceutically acceptable carrier, for example, cocoa butter, polyethylene glycol, polyoxyethylene sorbitan fatty acid ester surfactants, or lecitin.

In addition, the present invention provides a food composition containing an extract from a plant of the genus Agrimonia, which plays an auxiliary role in preventing and treating hepatic diseases. The food composition may be supplied as various kinds of foods, including functional foods, nutritional supplements, nutrients, pharmafoods, health foods, nutraceuticals, designer foods and food additives. It will be apparent to those of ordinary skill in the art that the food composition can be prepared in various food forms as described above according to common methods in the art. For example, health foods are drinkable in the form of tea, juice, jelly and health drinks, which are utilized as folk medicine. In addition, functional foods may be in the form of candy, yogurt, crackers, butter, margarine, and the like. The food additives may be in the form of powders or concentrated solutions. A suitable concentration of the Agrimonia extract contained in a food is preferably in the range from about 1 to about 5 mg per 100 g of food weight.

The present invention will be explained in more detail with reference to the following examples in conjunction with the accompanying drawings. However, the following examples are provided only to illustrate the present invention, and the present invention is not limited to them.

### EXAMPLE 1

### Preparation of Extracts from Plants of the Genus Agrimonia

After drying and then pulverizing the whole of Agrimonia eupatoria L., Agrimonia pilosa L., Agrimonia coreana N. and Agrimonia coreana N. for. pilosella Satake, 1 g of the pulverized products were mixed with 40 ml of distilled water, respectively, and extracted at 60° C. for 1 hour. Thereafter, each of the extract solutions was centrifuged at high speed, and the resulting supernatant was filtered through a 0.2 Am membrane to remove insoluble components, thus giving an extract. Dry weight of each extract was found to be 2.2 mg/ml of the supernatant.

### EXAMPLE 2

### Assay for Activities of Extracts from Plants of the Genus Agrimonia Against Production of Surface Antigens of HBV

### EXPERIMENTAL EXAMPLE 1

### Assay for an Activity of a Water-Soluble Extract from A. eupatoria L. against Production of Surface Antigens of HBV

2, 4 or 6 μl of the water-soluble extract (2.2 mg/ml) from Agrimonia eupatoria L. prepared in Example 1 was added to each well of a 96-well plate containing cultured human hepatoma cell line PLC/PRF/5, which synthesizes and secretes surface antigens of HBV (HBsAg), in which total volume of each well was 100 μl. After 48 hours of incubation, 100 μl of culture media was transferred to another 96-well plate, to which antibodies to surface antigens of HBV (HBsAb) were attached, and incubated at 37° C. for 1 hour. 25 μl of a solution of peroxidase-conjugated surface antibodies was added to each well, followed by incubation of 30 minutes. After being washed with phosphate buffer five times, each well was supplemented with a solution containing a substrate of peroxidase, and color development was then performed. Absorbance of the resulting solution was measured at 450 nm using ELISA reader. Culture medium not containing the water-soluble extract from A. eupatoria L. was used as a control group, while HBV-positive serum (P.S) and HBV-negative serum (N.S) were used as comparative groups.

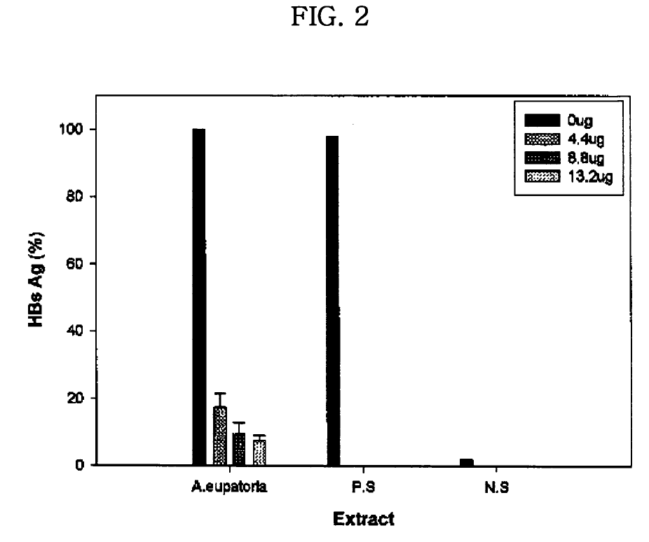

The results are given in FIG. 2. As shown in FIG. 2, the water-soluble extract from A. eupatoria L. was found to reduce hepatitis B surface antigen (HBsAg) levels in culture media of the hepatoma cell line PLC/PRF/5 in a dose-dependent manner.

### EXPERIMENTAL EXAMPLE 2

### Assay for an Activity of a Water-Soluble Extract from A. eupatoria L. Against Production of Envelope Antigens of HBV

2.2, 4.4 or 8.7 μl of the water-soluble extract (2.2 mg/ml) from Agrimonia eupatoria L. prepared in Example 1 was added to each well of a 96-well plate containing cultured HepG2 2.2.15 cells, in which total volume of each well was 100 μl. After 48 hours of incubation, 100 μl of culture media was transferred to another 96-well plate, to which antibodies to envelope antigens of HBV (HBeAb) were attached, and incubated at 37° C. for 1 hour. 25 μl of a solution of peroxidase-conjugated surface antibodies was added to each well, followed by incubation for 30 minutes. After being washed with phosphate buffer five times, each well was supplemented with a solution containing a substrate of peroxidase, and color development was then performed. Absorbance of the resulting solution was measured at 450 nm using ELISA reader. Culture medium not containing the water-soluble extract from A. eupatoria L. was used as a control group, while HBV-positive serum (P.S) and HBV-negative serum (N.S) were used as comparative groups.

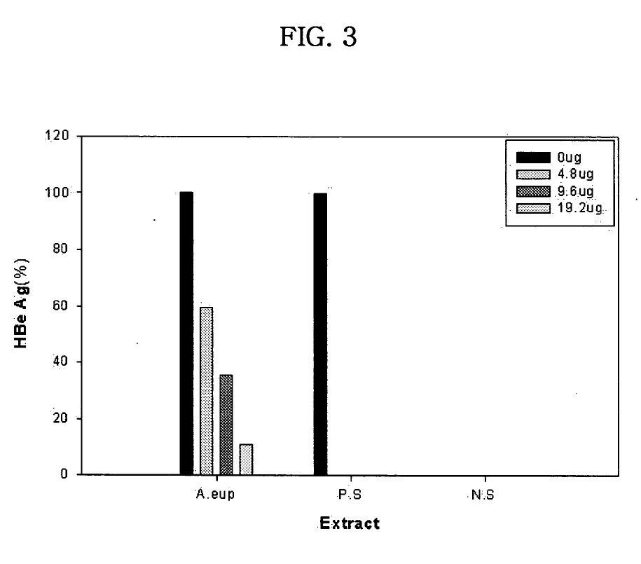

The results are given in FIG. 3. As shown in FIG. 3, the water-soluble extract from A. eupatoria L. was found to reduce hepatitis B envelope antigen (HBeAg) levels in culture media of HepG2 2.2.15 cells in a dose-dependent manner.

### EXPERIMENTAL EXAMPLE 3

### Assay for Activity of Water-Soluble Extract from A. eupatoria L. Against HBV Replication

After incubating HepG2 2.2.15 cells in culture dishes, the cultured HepG2 2.2.15 cells were treated with the water-soluble extract from A. eupatoria L. in various amounts of 25, 50, 100 and 200 μg, and incubated at 37° C. for 3 days. The culture medium was then centrifuged at 1,200 rpm for 10 min. The resulting supernatant was supplemented with 10% polyethylene glycol (PEG) by volume, incubated at 4° C. for 1 hour, and then centrifuged at 3,000 rpm for 15 minutes. The resulting pellet was suspended in a solution of 1% TNE (10 mM Tris-Cl, 100 mM NaCl, 1 mM EDTA), and phenol extraction and then phenol/chloroform extraction were performed to remove impurities. Viral DNA was then collected by adding 100% ethanol to the pellet and then centrifuging at 13,000 rpm for 10 minutes. The recovered viral DNA samples were electrophoresed on an agarose gel to analyze an effect of the extract from A. eupatoria L. on HBV-DNA levels.

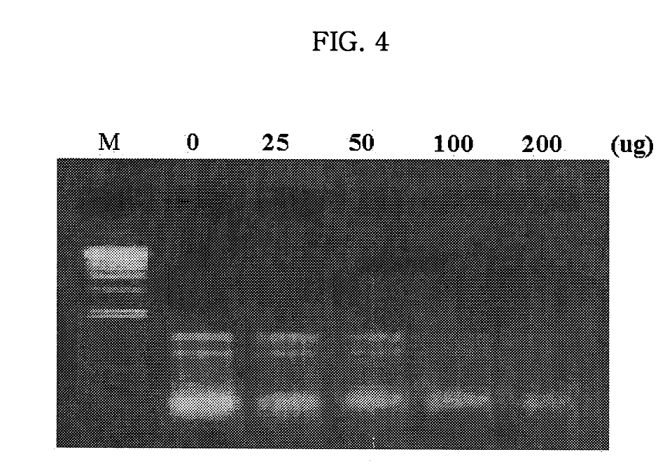

The results are given in FIG. 4. As shown in FIG. 4, the water-soluble extract from A. eupatoria L. was found to reduce HBV-DNA levels in a dose-dependent manner.

### EXPERIMENTAL EXAMPLE 4

### Assay for Activity of Water-Soluble Extract from A. eupatoria L. Against HBV Polymerase

1 ml of blood from hepatitis patients was mixed with 20% sucrose, and then centrifuged at 100,000× g for 16 hours. After discarding supernatant, the pellet was suspended in a mixture of 100 μl of 50 mM Tris and 50 μl of a buffer solution (100 mM Tris (pH 7.5), 300 mM NH₄Cl, 4 mM EDTA, 100 mM MgCl₂, 4% beta-mercaptoethanol, 2% NP-40). The resulting suspension was supplemented with 8 μl of each of dATP, dGTP and dTTP and 2 μl of ³²P-labelled dCTP, and then aliquoted into four tubes. To each tube, the water-soluble extract from A. eupatoria L. was added in various amounts of 0, 31.25, 62.5 and 125 μg, followed by overnight incubation at 37° C. The reaction mixture was reacted with 10 μl of proteinase K (10 mg/ml) at 37° C. for 2 hours to remove polymerase. Thereafter, isotope-labeled DNA was recovered by performing phenol extraction and then phenol/chloroform extraction to remove impurities, and then precipitating DNA using isopropanol. The precipitated DNA was washed with 70% ethanol, dried, and suspended in distilled water. The isotope-labeled DNA samples were electrophoretically separated on an agarose gel, and then transferred to a nitrocellulose (NC) membrane for 3 hours using a Transfer Kit. The resulting NC membrane was exposed to an X-ray film, which was then developed.

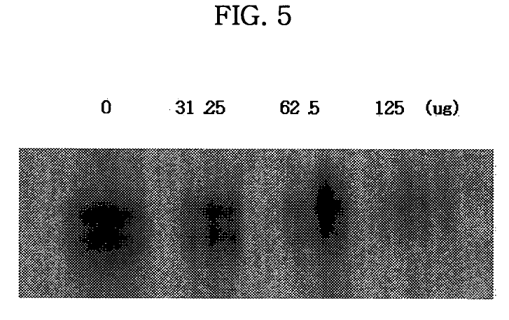

The results are given in FIG. 5. As shown in FIG. 5, the water-soluble extract from A. eupatoria L. was found to reduce newly synthesized HBV-DNA levels in a dose-dependent manner, thus indicating that the extract has an activity of suppressing HBV DNA polymerase activity.

### EXPERIMENTAL EXAMPLE 5

### Comparative Assay for Activities of Water-Soluble Extracts from Plants of the Genus Agrimonia Against Production of Surface Antigens of HBV

2 and 4 μl from the extract solutions (2.2 mg/ml) of each of A. eupatoria L., A. pilosa L., A. coreana N. and A. coreana N. for. pilosella Satake was added to wells of a 96-well plate containing cultured hepatoma cell line PLC/PRF/5, in which total volume of each well was 100 μl. After 48 hours of incubation, 100 μl of culture medium was transferred to another 96-well plate, to which antibodies to surface antigens of HBV (HBsAb) were attached, and incubated at 37° C. for 1 hour. 25 μl of a solution of peroxidase-conjugated surface antibodies was added to each well, followed by incubation for 30 minutes. After being washed with phosphate buffer five times, each well was supplemented with a solution containing a substrate of peroxidase, and color development was then performed. Absorbance of the resulting solution was measured at 450 nm using ELISA reader. Culture medium not containing the water-soluble extract from A. eupatoria L. was used as a control group.

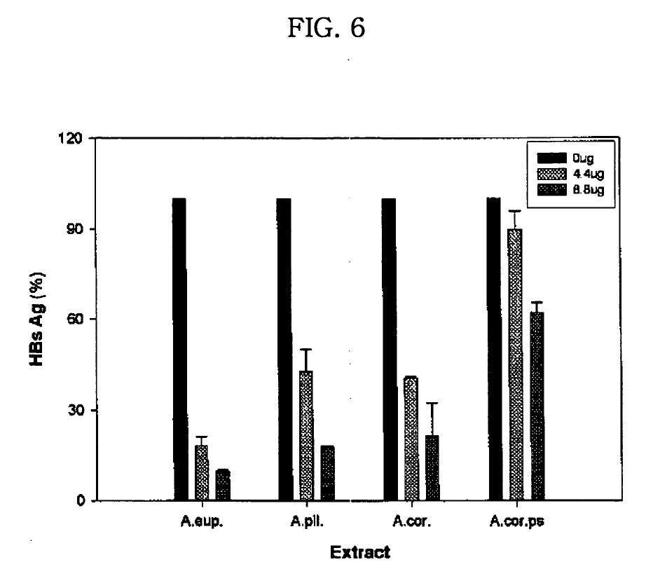

The results are given in FIG. 6. As shown in FIG. 6, all of the water-soluble extracts from the four species of the genus Agrimonia were found to reduce hepatitis B surface antigen (HBsAg) levels in culture media of the hepatoma cell line PLC/PRF/5 in a dose-dependent manner, while the extract from A. eupatoria L. showed the most potent activity in inhibiting production of HBsAg.

### EXAMPLE 3

### Effect of Extraction Temperature on Activity of Water-Soluble Extract from A. eupatoria L. Against Production of HBsAg

After drying and then pulverizing the whole of a plant A. eupatoria L., 1 g of the pulverized product was mixed with 40 ml of distilled water, and extracted at various temperatures of 37, 45, 55 and 60° C. for 1 hour. Thereafter, each of the extract solutions was centrifuged at high speed, and the resulting supernatant was filtered through a 0.2 μm membrane to remove insoluble components, thus giving an extract. Dry weight of each extract was found to be 2.2 mg/ml of the supernatant. 100 μl of culture medium containing 2, 4 or 6 μl of each of the water-soluble extracts (2.2 mg/ml) from A. eupatoria L. was added to each well of a 96-well plate containing incubated human hepatoma cell line PLC/PRF/5, in which total volume of each well was 100 μl. After 48 hours of incubation, 100 μl of culture medium was transferred to another 96-well plate, to which antibodies to surface antigens of HBV (HBsAb) were attached, and incubated at 37° C. for 1 hour. 25 μl of a solution of peroxidase-conjugated surface antibodies was added to each well, followed by incubation for 30 minutes. After being washed with phosphate buffer five times, each well was supplemented with a solution containing a substrate of peroxidase, and color development was then performed. Absorbance of the resulting solution was measured at 450 nm using ELISA reader. Culture medium not containing the water-soluble extract from A. eupatoria L. was used as a control group.

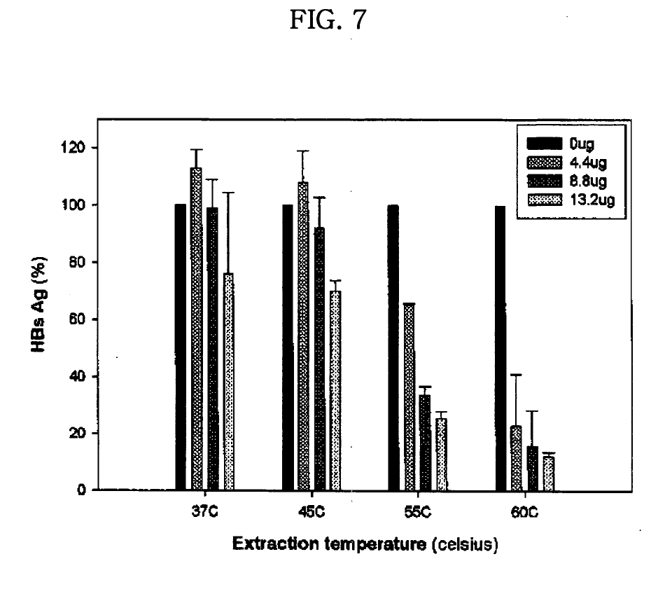

The results are given in FIG. 7. As shown in FIG. 7, the water-soluble extract from A. eupatoria L. was found to have a remarkably increased effect in inhibiting production of HBsAg when being extracted at a temperature of 55° C. or higher.

### EXAMPLE 4

### Effect of Extraction Time on Activity of Water-Soluble Extract from A. eupatoria L. Against Production of HBsAg

After drying and then pulverizing the whole of A. eupatoria L., 1 g of the pulverized product was mixed with 40 ml of distilled water, and extracted at 25° C. for 3, 5 and 7 days. Thereafter, each of the extract solutions was centrifuged at high speed to remove insoluble components. 2 or 4 μl of each of the water-soluble extracts (2.2 mg/ml) from A. eupatoria L. was added to wells of a 96-well plate containing incubated human hepatoma cell line PLC/PRF/5, in which total volume of each well was 100 μl. After 48 hours of incubation, 100 μl of culture medium was transferred to another 96-well plate, to which antibodies to surface antigens of HBV (HBsAb) were attached, and incubated at 37° C. for 1 hour. 25μl of a solution of peroxidase-conjugated surface antibodies was added to each well, followed by incubation for 30 minutes. After being washed with phosphate buffer five times, each well was supplemented with a solution containing a substrate of peroxidase, and color development was then performed. Absorbance of the resulting solution was measured at 450 nm using ELISA reader. Culture medium not containing the water-soluble extract from A. eupatoria L. was used as a control group.

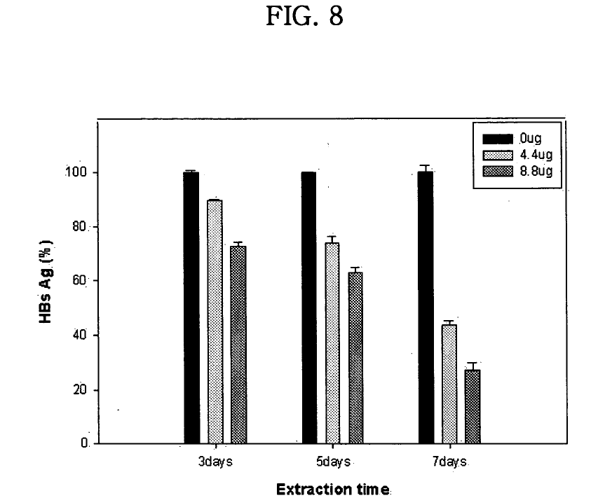

The results are given in FIG. 8. As shown in FIG. 8, the water-soluble extract from A. eupatoria L. was found to have a remarkably increased effect in inhibiting production of HBsAg when being extracted for 7 days at 25° C.

### EXAMPLE 5

### Assay for Activities of Fractions Obtained by Extraction of A. eupatoria L. Using Organic Solvents Against Production of HBsAg

According to an extraction process as shown in FIG. 1, the whole plant of A. eupatoria L. (10.015 g) was extracted with methylalcohol. The resulting extract was treated with an equivalent volume of the organic solvent hexane to separate into a hexane fraction (0.145 g) and an aqueous fraction. The aqueous fraction obtained from the hexane treatment was again treated with an equivalent volume of chloroform, yielding a chloroform fraction (0.329 g) and an aqueous fraction. The aqueous fraction obtained from the chloroform treatment was treated with an equivalent volume of butylalcohol, producing a butylalcohol fraction (0.807 g) and an aqueous fraction (0.462 g). Thereafter, each fraction was dried completely, and used in the following experiment after being dissolved in a methylalcohol solution.

Hepatoma cell line HepG2 2.2.15, cultured in a 96-well plate, was treated with 2, 5 and 10 μg of each fraction for 48 hours. Thereafter, 100 μl of culture medium was transferred to another 96-well plate, to which antibodies to surface antigens of HBV (HBsAb) were attached, and incubated at 37° C. for 1 hour. 25 μl of a solution of peroxidase-conjugated surface antibodies was added to each well, followed by incubation of 30 minutes. After being washed with phosphate buffer five times, each well was supplemented with a solution containing a substrate of peroxidase, and color development was then performed. Absorbance of the resulting solution was measured at 450 nm using ELISA reader. Culture medium not containing the fraction obtained from A. eupatoria L. was used as a control group.

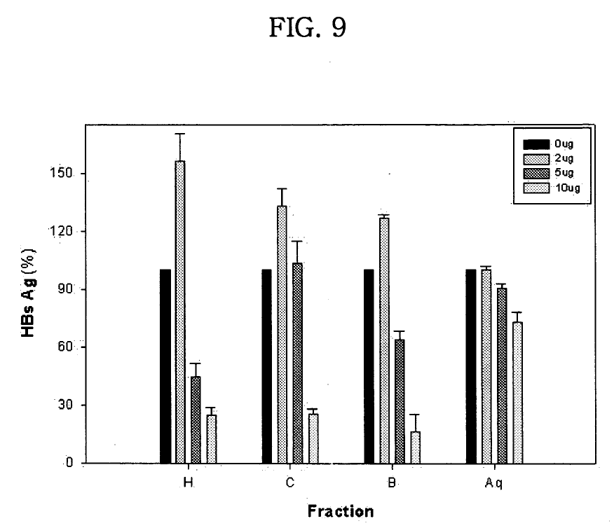

The results are given in FIG. 9 in which H, C, B and Aq mean the hexane fraction, the chloroform fraction, the butylalcohol fraction and the aqueous fraction, respectively. As shown in FIG. 9, each fraction of the organic solvents was found to have higher activity than the aqueous fraction in reducing HBsAg levels.

### EXAMPLE 6

### Assay for Activities of Fractions Obtained by Extraction of A. eupatoria L. Using Organic Solvents Against Production of HBeAg

The hexane, chloroform, butylalcohol and aqueous fractions of A. eupatoria L. prepared in Example 5 were added to each well of a 96-well plate containing cultured HepG2 2.2.15 cells in various amounts of 2, 5 and 10 μg, followed by incubation of 48 hours. Thereafter, 100 μl of culture medium was transferred to another 96-well plate, to which antibodies to envelope antigens of HBV (HBeAb) are attached, and incubated at 37° C. for 1 hour. 25 μl of a solution of peroxidase-conjugated envelope antibodies was added to each well, followed by incubation of 30 minutes. After being washed with phosphate buffer five times, each well was supplemented with a solution containing a substrate of peroxidase, and color development was then performed. Absorbance of the resulting solution was measured at 450 nm using ELISA reader. Culture medium not containing the fraction obtained from A. eupatoria L. was used as a control group.

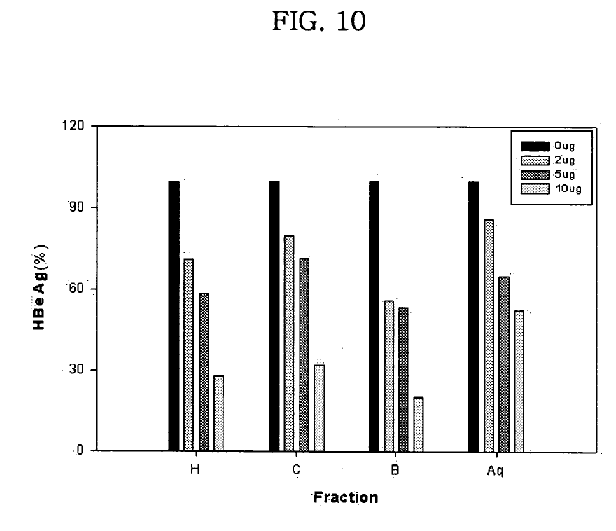

The results are given in FIG. 10. As shown in FIG. 10, the hexane fraction (H), chloroform fraction (C) and butylalcohol fraction (B) were found to have higher activity than the aqueous fraction (Aq) in reducing HBeAg levels.

### Industrial Applicability

As described hereinbefore, the extracts from plants of the genus Agrimonia according to the present invention, especially the extracts from the species A. eupatoria L., have excellent effects in preventing and treating hepatic diseases.

What is claimed is:

1. A pharmaceutical composition for preventing or treating hepatic disease, comprising a pharmaceutically effective amount of an extract from a plant Agrimonia eupatoria L.

2. The pharmaceutical composition as set forth in claim 1, wherein the extract is water-soluble.

3. The pharmaceutical composition as set forth in claim 1, wherein the hepatic disease is hepatitis B, liver cirrhosis or hepatocellular carcinoma.

4. A food composition for treating patients suffering from hepatic diseases, comprising an extract from a plant Agrimonia eupatoria L. as an effective ingredient.

5. A method of preparing a water-soluble extract from a plant Agrimonia eupatoria L. having improved efficacy in preventing or treating hepatic diseases, which comprises the steps of:
pulverizing a plant Agrimonia eupatoria L.;
mixing the pulverized product with water;
heating the mixture to give an extract solution; and
removing water-insoluble components from the extract solution to produce a water-soluble extract.

6. The method as set forth in claim 5, wherein the mixture of the pulverized product and water is heated at about 55° C. or more.

7. The method as set forth in claim 5, wherein the mixture of the pulverized product and water is heated at about 20° C. to 30° C. for about 7 days or more.

8. A method of preparing a water-soluble extract from a plant of the genus Agrimonia having improved efficacy in preventing or treating hepatic diseases, which comprises the steps of:
pulverizing a plant of the genus Agrimonia;
mixing the pulverized product with water;
heating the mixture to give an extract solution at about 55° C. or more; and
removing water-insoluble components from the extract solution to produce a water-soluble extract.

9. The method as set forth in claim 8, wherein the mixture of the pulverized product and water is heated at about 55° C. to 65° C. for about 50 to 90 minutes.

10. A method of preparing a water-soluble extract from a plant of the genus Agrimonia having improved efficacy in preventing or treating hepatic diseases, which comprises the steps of:
pulverizing a plant of the genus Agrimonia;
mixing the pulverized product with water;
heating the mixture to give an extract solution at about 20° C. to 30° C. for about 7 days or more; and
removing water-insoluble components from the extract solution to produce a water-soluble extract.

11. The method as set forth in claims 8 or 10, wherein the plant of the genus Agrimonia is one or more selected from the group consisting of Agrimonia eupatoria L., Agrimonia pilosa L., Agrimonia coreana N., Agrimonia coreana N. for. pilosella Satake, Agrimonia gryposepala, Agrimonia rostellata, Agrimonia pubescens, Agrimonia parviflora, Agrimonia striata, Agrimonia ordorata, Agrimonia incisa, Agrimonia polyphylla, Agrimonia microcarpa, Agrimonia bracteata, Agrimonia repens, Agrimonia platycarpa, Agrimonia pumila, and Agrimonia asiatica.

12. A pharmaceutical composition for preventing or treating a hepatic disease, comprising a water-soluble extract from a plant of the genus Agrimonia prepared according to the method of claims 8 or 10.

13. The pharmaceutical composition as set forth in claim 12, wherein the hepatic disease is hepatitis B, liver cirrhosis or hepatocellular carcinoma.

14. A food composition for patients suffering from hepatic diseases, comprising a water-soluble extract from a plant of the genus Agrimonia prepared according to the method of claims 8 or 10.

15. A method of preparing an organic solvent-soluble extract from a plant of the genus Agrimonia having improved efficacy in preventing or treating hepatic diseases, which comprises the steps of:
extracting a plant of the genus Agrimonia with an organic solvent; and
removing an aqueous fraction to obtain an organic solvent-soluble fraction.

16. The method as set forth in claim 15, wherein the plant of the genus Agrimonia is one or more selected from the group consisting of Agrimonia eupatoria L., Agrimonia pilosa L., Agrimonia coreana N., Agrimonia coreana N. for. pilosella Satake, Agrimonia gryposepala, Agrimonia rostellata, Agrimonia pubescens, Agrimonia parviflora, Agrimonia striata, Agrimonia ordorata, Agrimonia incisa, Agrimonia polyphylla, Agrimonia microcarpa, Agrimonia bracteata, Agrimonia repens, Agrimonia platycarpa, Agrimonia pumila, and Agrimonia asiatica.

17. A pharmaceutical composition for preventing or treating a hepatic disease, comprising an organic solvent-soluble extract from a plant of the genus Agrimonia prepared according to the method of claim 15.
